(19) United States
(12) Patent Application Publication
(10) Pub. No.: US 2023/0062584 A1
(43) Pub. Date: Mar. 2, 2023

(54) Risk Score and Classification System for Prevention of Complications in Plastic Surgery

(71) Applicant: AIRA10 MEDICAL LLC, Albuquerque, NM (US)

(72) Inventors: Williams Erik Bukret, Buenos Aires (AR); Karina Alejandra Pesce, Buenos Aires (AR)

(21) Appl. No.: 17/888,035

(22) Filed: Aug. 15, 2022

Related U.S. Application Data
(60) Provisional application No. 63/240,172, filed on Sep. 2, 2021.

(51) Int. Cl. G16H 50/30; G16H 50/20; G16H 10/20

(52) U.S. Cl. CPC G16H 50/30; G16H 10/20; G16H 50/20

(57) ABSTRACT

A real-time method for risk assessment and prediction of complications in plastic surgery. It includes a scoring method that classifies patients in distinct levels or risk-groups according to a risk score. According to the variables present in each individual, the algorithm estimates a risk factor for that particular data set provided by the patient through a questionnaire, calculates a risk score, classifies each patient in a risk group, and displays results in different electronic devices and personalized apps.

### CROSS REFERENCE TO RELATED APPLICATION

This application claims the benefit of U.S. Provisional Pat. Application No. 63/240,172, filed Sep. 2, 2021, which is incorporated by reference herein in its entirety.

### INCORPORATION BY REFERENCE PURSUANT TO 37 C.F.R. §1.57(B

The article: "Pannucci, C. et.al (2016). Benefits and Risks of Prophylaxis for Deep Venous Thrombosis and Pulmonary Embolus in Plastic Surgery. Plastic and Reconstructive Surgery, 137(2), 709-730. doi: 10.1097/01.prs.0000475790.54231.28,," attached to this specification as a Specification Annex, is incorporated by reference herein in its entirety.

### TECHNICAL FIELD

This invention relates generally to a multifactor risk prevention and scoring system and method for surgical procedures and, more particularly, to a computerized system and method for predicting complications after cosmetic surgeries based on a multifactorial analysis, allowing patients to be classified into different risk groups.

### BACKGROUND OF THE INVENTION

Prevention of complications is one of the most important concerns of plastic surgeons since it implies reducing the costs of hospitalizations, treatments, reoperations and also increasing the degree of patient satisfaction. Thus, it is necessary to have an efficient universal system that serves for risk analysis and the prevention of complications. Currently, the recommendations of the expert consensus guidelines are used for prevention and there is no model that can predict complications in real-time, taking into account the risk factors of each patient. However, these proposed norms and parameters vary according to the authors and should be updated based on evidence. Furthermore, these guidelines are not practical when evaluating patients because they often do not apply to particular cases, which makes it difficult to adequately classify patients according to their risk.

According to the guide of recommendations of the American Society of Plastic Surgery of the year 2011, which is still in force, high-risk patients are classified as those with a body mass index ≥ 35, age ≥ 50 years, liposuction greater than 5000 ml, and combined surgeries particularly abdominoplasty with liposuction. Later in 2018, Dr. Rod Rohrich suggested a change in the classification parameters of high-risk patients: body mass index ≥ 30, age ≥ 40 years, liposuction greater than 3 liters (Table 1).

Table 1

Recommendations for prevention of complications.
Recommendations

| | | |
|---|---|---|
| | ASPS¹ | Rohrich, R² |
| Age | >50 | >40 |
| BMI | >40 | >30 |
| Lipoaspirate | >5000 ml | >3000 ml |
| Combined surgeries | Especially abdominoplasty with liposuction | |

BMI, body mass index. ASPS American Society of Plastic Surgeons; ASPS Patient
Safety Committee. Pathways to preventing adverse events in ambulatory surgery in
2011.Rohrich RJ, Mendez BM, Afrooz PN. An update on the safety and efficacy of outpatient plastic surgery: A review of 26,032 consecutive cases. Plast Reconstr Surg. 2018, (4):902-908.

However, at present, there is no availability of a risk prediction model that adjusts to variations in the mentioned risk factors. For this reason, providing a method and system for predicting complications after plastic surgery based on a classification of patients in different risk groups in consideration of a multiplicity of risk factors and their variations fulfills a long felt yet unresolved need, substantially advancing the field.

### SUMMARY OF THE INVENTION

The main object of the present invention is to provide a computing system for establishing a predictive score for the risk of complications after plastic surgery, and for classifying patients into different risk groups. For this reason, a multifactorial correlation analysis was performed between risk factors and complications to generate an evidence-based multifactorial classification system. In preliminary studies, the relationship and relative risk were established between several independent risk factors and complications. Consequently, it is still necessary to establish the role of some risk factors such as smoking, sex, combined surgeries, as well as the combination of risk factors in a predictive model to establish an efficient risk analysis model. Thus, the present invention considers the risk factors of each patient individually, classifies patients according to their risk of suffering complications through a scoring system, thus determining the probabilities of suffering the most common complications after plastic surgery. Moreover, the proposed system classifies patients in real-time into different risk groups based on their risk score. Another advantage of the system is that it allows personalized recommendations to reduce risk, improving the eligibility of the patient and the appropriate surgical environment according to their risk. Overall, the risk assessment system does not intend to replace plastic surgeon evaluation but augment it by facilitating the collection of data, avoiding errors, optimizing time, and reducing cost. Another advantage is that the information is always available and displayed on multiple devices.

A computing system for establishing a predictive score for the risk of complications after plastic surgery in accordance with the present invention, is achieved by combining some or all of the following aspects:
a) A computer or electronic device comprising a processor and a memory and connected to internet
b) An online evaluation form accessible through a patient interface that the patient can access through a computer or electronic device comprising a processor and a memory and connected to internet. This form includes input-form-fields to enter patient information, medical antecedents, and risk factors. All the data needed to calculate the Caprini Score of the patient should be recollected in this step as well as the cigarette consumption, BMI (or height and weight) and age of the patient.
c) A database in which the information from point b is collected, stored, and from which it can be retrieved as required
d) An automated risk-score calculation algorithm based on the information stored on database c, using the following formula to calculate the Risk Score of the patients:
$$\text{Risk Score} = {\text{W}}_{1} {\text{f}}_{1} + {\text{W}}_{2} {\text{f}}_{2} + {\text{W}}_{3} {\text{f}}_{3} + {\text{W}}_{4} {\text{f}}_{4}$$

Where W1, W2, W3, and W4 are variables of the formula and f1, f2, f3, and f4 are factors of the formula, defined as:
W₁ is the age of the patient, W₂ is the BMI of the patient, W₃ is the Caprini Score of the patient, W₄ is the number of cigarettes the patient smokes per week, f₁ is 100^{∧}(-3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more.
e) A risk classification algorithm to assign a Risk group to the patients based on the Risk Score calculated in point d, as low risk (1 to <1.2 points), moderate risk (≥1.2 to <1.4 points), and high risk (≥1.4 points) of complications.
f) A deployment of information process to display the results through a Physician interface in a computer or electronic device comprising a processor and a memory and connected to the Internet.
g) A system for adjusting the values of the Formula of point (d) by fine-tuning the variables used and the values of the factors for which they are multiplied, by collecting clinical data, performing statistical analysis of data, using Pearson’s correlation coefficient test and p-value to find risk factors with statistically significant correlation with complications, performing the Pearson correlation coefficient test and the p-value between the risk score and the risk factors to determine which are independent predictors, establishing a predictive scoring system based on the results of the multifactorial correlation analysis, as a Coefficient of multiple correlation, and validating the scoring system by machine learning using a vector classification system (VCS).
h) A reassessment mechanism to keep the information in the database up to date.
and
i) The computing system is embodied in a computer having a processor, a memory, a screen, and internet access.

A method to establish a scoring system and classification into risk groups for predicting the risk of complications after plastic surgeries is disclosed, which includes the following steps:
(1) Collecting clinical data from the patients, including all the information needed to calculate the Caprini Score of the patient as well as the cigarette consumption, BMI (or height and weight) and age of the patient, through an online evaluation form
(2) Storing the data from step 1 on a database
(3) Calculating the Caprini Score of the Patient and the BMI based on height and weight. A Person of Ordinary Skill in the art is aware of how to calculate a Caprini Score. However, to avoid any possible misinterpretation, the article "Pannucci, C. et.al (2016). Benefits and Risks of Prophylaxis for Deep Venous Thrombosis and Pulmonary Embolus in Plastic Surgery. Plastic and Reconstructive Surgery, 137(2), 709-730. doi: 10.1097/01.prs.0000475790.54231.28," is attached to the submission of the present application and hereby incorporated by reference in its entirety.
(4) Calculating the Risk Score of the patient using the formula:

“Risk score = W₁ f₁ + W₂ f₂+ W₃ f₃+ W₄ f₄ = 1- 2.5” to the information collected from the patient, where W₁ is the age of the patient, W₂ is the BMI of the patient, W₃ is the Caprini Score of the patient, W₄ is the number of cigarettes the patient smokes per week, f₁ is 100^{∧}(-3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more.
(5) Classifying the Patients in Risk Groups according to the algorithm:
Risk of complications: low risk when the Risk score is (1 to <1.2 points), moderate risk when the Risk score is (≥1.2 to <1.4 points), and high risk when the Risk score is (≥1.4 points).
and
(6) Displaying the Risk Score calculated on step 4 and the risk group calculated on step 5, together with the clinical data collected on step 1, though a Physician’s interface in a computer or electronic device comprising a processor and a memory and connected to the Internet.

### BRIEF DESCRIPTION OF THE DRAWINGS

FIG. 1 is a conceptual flow diagram of an embodiment of a system for establishing a predictive score for the risk of complications after plastic surgery in accordance with the present invention.

FIG. 2 is a conceptual flow diagram of the data management process in an embodiment of a system for establishing a predictive score for the risk of complications after plastic surgery in accordance with the present invention.

FIG. 3 is a table containing data collected from patients in an embodiment of a system for establishing a predictive score for the risk of complications after plastic surgery in accordance with the present invention.

### DETAILED DESCRIPTION AND BEST MODE OF IMPLEMENTATION

Disclosed is a system for establishing a predictive score for the risk of complications after plastic surgery, and for classifying patients into different risk groups. A research study was performed to evaluate the risk factors predictive of complications and validate the new assessment system by a machine learning process. In it, a retrospective analysis was performed with 372 patients operated on by the author between 2015 and 2020 to ensure the consistency of the results for internal validation. Pearson’s correlation test was used for the analysis of risk factors and complications. The difference between the mean of the risk scores of the three risk groups was assessed using one-way analysis of variance (ANOVA). For the analysis of the incidence of risk factors and relative risk of complications after cosmetic surgeries, systematic reviews and meta-analyzes, multicentric analyzes, and evidence-based research studies classified as type 1 and 2 were included, with more than 1000 cases in the last 5 years. The incidence of independent factors that exhibited a statistically significant positive correlation (p <0.05) between risk factors and complications and the relative risk (RR) was analyzed. Each risk factor was assigned a weighted coefficient (f) to stratify the risk of complications. Subsequently, a weighted sum method with the relevant risk factors (W) was used to create a risk scale from 1 to 2.5. The minimum risk was assigned the number 1 to obtain a minimum multiplication factor since any surgery involves some risk that is different from 0.

The clinical data obtained from the patients, including the independent risk factors, are personal data of the patient, age, sex, weight, height, smoking, previous surgeries, medical history, pathological history, requested surgery, and body measurements. The indexes used by the algorithm are calculated by the system from the data supplied by the patients: body mass index, Caprini score, body dysmorphic syndrome detection test, etc. Then the model calculates the risk score according to the data, classifying to which risk groups each patient belongs using the mentioned formula.

A simplified scoring system is established based on the weighted sum model. The risk score is a weighted sum method assisted by a narrow Al, expressed mathematically by the following formula:

$$\text{Risk Score} = {\text{W}}_{1} {\text{f}}_{1} + {\text{W}}_{2} {\text{f}}_{2} + {\text{W}}_{3} {\text{f}}_{3} + {\text{W}}_{4} {\text{f}}_{4} = 1 - \text{2} \text{.5}$$

where W₁ is the age of the patient, W₂ is the BMI of the patient, W₃ is the Caprini Score of the patient, W₄ is the number of cigarettes the patient smokes per week, f₁ is 100^(-3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more.

The system automatically classifies patients into one of the three risk groups according to their scores, such as A = low risk (1 to <1.2 points), B = moderate risk (≥1.2 to <1.4 points) and C = high risk (≥ 1.4 points) of complications.

Table 2

Risk score and risk group classification

| | | |
|---|---|---|
| Risk groups | Score | Risk |
| A | 1 to <1.2 | Low |
| B | ≥1.2 to <1.4 | Moderate |
| C | ≥1.4 to <2.5 | High |

The classification and scoring model were validated through machine learning in Python language using a support vector classification process (SVC). The predictive algorithm included the following risk factors are selected based on their relative importance and statistical significance (p <0.01): age, BMI, Caprini score, and smoking. The set of 372 data cases was divided into 2 samples. A training sample (0.80) and a test sample (0.20) with a random seed of 100 cases. Patients with missing data were excluded from the data set. All procedures performed in this study were performed in accordance with the Declaration of Helsinki. All patient information was disidentified and retrospective.

The frequency of cases, complications, odds ratios (OR) and the mean risk score in each risk group were as follows: low risk, 215 cases (9 complications, mean = 1.04, odds ratio [OR] = 0, 04, 99% confidence interval [Cl] = 1.045-1.035); moderate risk, 113 cases (9 complications, mean = 1.27, OR = 0.09, 99% Cl = 1.277-1.263); and high risk, 44 cases (10 complications, mean = 1.67, OR = 0.29, 99% Cl = 1.673-1.627). The difference between the means of the risk groups assessed with ANOVA was the following: f-value = 1461.2, p-value = 4.54828884e-176 (Table 3).

Table 3

Frequency of cases, complications, mean and Odd Ratio per group (n = 372)

| Risk group | Cases | Complications | Mean | OR |
|---|---|---|---|---|
| Low | 215 | 9 | 1.04 | 0.04 |
| Moderate | 113 | 9 | 1.27 | 0.09 |
| High | 44 | 10 | 1.65 | 0.29 |

OR odd ratio

The measures used to evaluate the performance of the model were the following: accuracy score, precision (positive predictive value), recall (sensitivity), and harmonic mean of precision and recovery (f1 score or Sørensen-Dice coefficient).

The accuracy score for SVC prediction was 100% in the training sample and 97.3% in the test sample. The positive predictive value of the method was 0.98, the sensitivity showed a weighted average of 0.97 and the f₁ score showed a weighted average of 0.97 (Table 4).

Table 4

Model Performance per Risk Group

| | Precision | Recall | f1-score | Support |
|---|---|---|---|---|
| Low | 1.00 | 0.83 | 0.91 | 6 |
| Moderate | 1.00 | 0.98 | 0.99 | 48 |
| High | 0.91 | 1.00 | 0.95 | 21 |
| Accuracy | | | 0.97 | 75 |
| Weighted avg | 0.98 | 0.97 | 0.97 | 75 |

Precision, positive predictive value; Recall, sensitivity; F1-score, harmonic mean of the precision and recall; avg, average

A cloud data collection system comprises an online form, a database, a spreadsheet for data collection and analysis, and a result display system for several applications. The data analysis system can calculate the risk of complications and classify patients into different risk groups according to their score obtained based on the information provided by the data entry system, and the visualization system will display the results in different custom user interfaces (UI). Compared with the expert committee recommendation guidelines used so far, the present invention selected for the first time the independent risk predictors of complications after plastic surgery used Pearson’s correlation coefficients to assign values to each risk factor, establish a risk prediction scoring system, and classified patients into risk groups. The performance score of the method evaluated by the accuracy score yields a value of 97.3%, which exhibits excellent predictive capacity. This system is easy to implement in daily practice, allowing for the first time a personalized evaluation in real-time, which allows improving the recommendation and education of patients to correct risk factors and choose the most appropriate surgical setting according to the risk of each patient.

To understand in a comparative way the difference in the probability of occurrence of complications in the different risk groups, we calculated the relative difference between groups. The relative difference in complications was 6.7 times greater in the high-risk group (RR, 0.29) and 2 times greater in the moderate-risk group (RR, 0.09) than in the low-risk group (RR, 0.04) (Table 5).

Table 5

Frequency of complications, relative risk, and relative difference per group (n = 372)

| Risk group | Cases | Complications | Mean | RR | RD |
|---|---|---|---|---|---|
| Low | 215 | 9 | 1.04 | 0.04 | |
| Moderate | 113 | 9 | 1.27 | 0.09 | 2.0 |
| High | 44 | 10 | 1.65 | 0.29 | 6.7 |

The variables selected to be included in the predictive model are statistically significant variables. The correlation table of significant variables is as follows (Table 6). Smoking was also included in the predictive model based on evidence and expert recommendations

Table 6

Correlations of risk score with risk factors

| | Risk score | BMI | Age | Caprini score |
|---|---|---|---|---|
| Risk score | 1.00 | 0.99 | 0.97 | 0.98 |
| BMI | 0.99 | 1.00 | 0.94 | 0.95 |
| Age | 0.97 | 0.94 | 1.00 | 0.99 |
| Caprini score | 0.98 | 0.95 | 0.99 | 1.00 |

### Establishment of a Scoring System to Predict Complications

Based on the results of the multivariate analysis, a prediction model for complications was established. The formula is as follows:

$$\text{Risk Score} = \text{W1} \text{f1} + \text{W2} \text{f2} + \text{W3} \text{f3} + \text{W4} \text{f4} = 1 - \text{2} \text{.5}$$

According to the data entered into the system, the assignment of the coefficients used in the algorithm will be (Table 7):

Table 7

Assignment of coefficients
Coefficients (f)

| f₁ | N° / [100^3] | |
|---|---|---|
| f2 | ≥25 = 0.2 | ≥30 = 0.4 |
| f3 | N^{o} * 0,245.00 | |
| f4 | <7 = 0.1 | ≥7 = 0.3 |

According to the scoring system, patients are classified into risk groups according to their scores, from which risk-adjusted recommendations are made. The relative risk of complication is significantly different in each group as was demonstrated by the ANOVA score and relative difference. Table 8.

Table 8

Risk group and recommendations according to score

| | | | |
|---|---|---|---|
| Risk group | Score | Risk | Recommendation |
| A | 1 a <1.2 | Low | In conditions |
| B | ≥1.2 a <1.4 | Moderate | Defer surgery |
| C | ≥1.4 a <2.5 | High | Defer surgery |

### The Predictive Value of the Model

For the validation of the predictive model with supervised machine learning, a database of 372 cases was used, which was divided into training (0.8) test (0.20) with a random seed of 100 cases. The performance metric of the chosen model was the accuracy score, the average accuracy between groups was 0.973, which indicates that the scoring system has a great predictive capacity, especially when the score is ≥ 1.2, since in the moderate and high-risk groups the accuracy reached 100% (Table 9).

Table 9

Model Performance per Risk Group

| | Precision | Recall | f1-score | Support |
|---|---|---|---|---|
| Low | 1.00 | 0.83 | 0.91 | 6 |
| Moderate | 1.00 | 0.98 | 0.99 | 48 |
| High | 0.91 | 1.00 | 0.95 | 21 |
| Accuracy | | | 0.97 | 75 |
| Weighted avg | 0.98 | 0.97 | 0.97 | 75 |

Precision, positive predictive value; Recall, sensitivity; F1-score, harmonic mean of the precision and recall; avg, average

Additionally, a risk-adjusted price (RAP) can be added to the system, to measure a surgery price after taking into account the degree of risk given by the risk score. Therefore, RAP risk-adjusted price is a number that varies linked to the risk score and will change according to the case. Mathematically is explained as a multiplier of the risk factor by the surgery price: E.g. (surgery price) times (risk factor) = U$D 2000 x 1.03 = 2060. The surgery prices must be provided by the user as a price list, so the app will take that information to calculate the RAP for each patient. The RAP is displayed in another column of the list, same as all other information for each patient. The purpose of risk-adjusted prices is to help doctors cover losses for future re-operations, treatment costs, and legal expenses associated with a higher risk of complications and at the same time to encourage patients to take action to reduce risk modifying risk factors before surgery, in other words, to actively participate in prevention.

Some general aspects of the present invention have been summarized so far in the first part of this detailed description and in the previous sections of this disclosure. Hereinafter, a detailed description of the invention as illustrated in the drawings will be provided. While some aspects of the invention will be described in connection with these drawings, it is to be understood that the disclosed embodiments are merely illustrative of the invention, which may be embodied in various forms. The specific materials, methods, structures, functional details, and scope disclosed herein are not to be interpreted as limiting. Instead, the intended function of this disclosure is to exemplify some of the ways -including the presently preferred ways- in which the invention, as defined by the claims, can be enabled for a Person of Ordinary Skill in the Art. Therefore, the intent of the present disclosure is to cover all variations encompassed within the spirit and scope of the invention as defined by the appended claims, and any reasonable equivalents thereof.

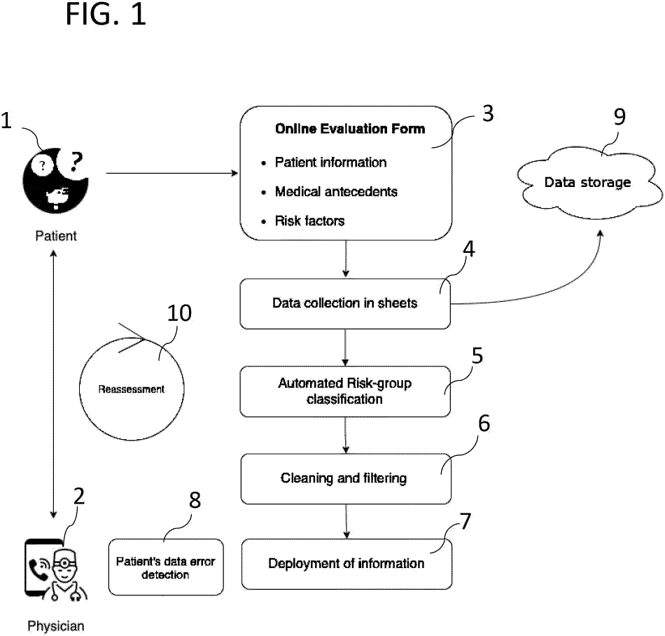

Referring to the drawings in more detail, FIG. 1 illustrates an embodiment of a system for establishing a predictive score for the risk of complications after plastic surgery in accordance with the present invention. In it, a Patient 1 introduces information to the system, which will be later retrieved by a Physician 2. This information is introduced by the Patient 1 through an Online Evaluation Form 3, which prompts the Patient 1 to submit self-assessed data about their personal information, medical antecedents, and risk factors, including all the information needed to calculate the Caprini Score of the patient as well as the cigarette consumption, BMI (or height and weight) and age of the patient, through an online form having checklists, multiple choice, dropdown menus radio buttons and/or similar formats for targeted data entry in which the relevant variables are considered.

The information collected through the Evaluation Form 3 can include items such as: Date, Email, Name, Last name, Age, BMI 36, Caprini Score, Price factor, Budget, Smoking (number of cigarettes consumed per week), Drugs, Alcohol, Venous thrombosis in legs or thighs (blood clot,) Diabetes, High blood pressure, Acute myocardial infarction, Ischemic heart disease, Vascular pathology, Cerebrovascular accident, Cardiac arrhythmia, Cancer, Thyroid, Allergies, Medications, Contraceptives (pills or patches), Inherited or family diseases (thrombosis, stroke, vasculopathy), Clotting or bleeding problems (with hematological diagnosis, e.g., factor deficit), previous surgery >1 month, Homocysteine, Surgery of interest, When would you like to have surgery?, Weight in Pounds, Height in inches, Pregnancies, Childbirth, C section, Number of abortions, Last menstruation date, Previous surgeries, Reoperation: The same surgery you require, Previous plastic surgeries, Asthma, Abdominal hernia, Stress in the last 3 months. It may be loss of a relative or job, separation, move, or economic factor, Covid 19 (any positive test), Gastric or duodenal ulcer (diagnosed by endoscopy), Chronic infectious diseases (Hepatitis B or C, HIV), Body Dysmorphic Disorder Questionnaire (BDDQ) based on DSM-IV diagnostic criteria for BDD, Are you overly concerned about the appearance of some part(s) of your body that you consider especially unattractive? Do these concerns preoccupy you? That is, do you think about them a lot and wish you could think about them less? Has your defect(s) caused you a lot of distress, torment, or pain? Has your defect(s) significantly interfered with your social life? Has your defect(s) significantly interfered with your schoolwork, your job, or your ability to function in your role? Are there things you avoid because of your defect(s)? How much time do you spend thinking about your defect(s) per day on average? ≥1h Is your main concern with your appearance that you aren’t thin enough or that you might become fat? Times you breastfed, Number of previous breast surgery, Size of the implants you have or the closest number (only in case of previous surgery) Chest circumference measured in centimeters at the level of the nipples, Chest circumference measured in centimeters at the level of the nipples you want to have (which you want to increase approximately or bodice measurement), Circumference measured in centimeters of the chest at the level of the submammary sulcus, just below the breasts, Abdomen circumference at the level of the navel, measured in centimeters. Abdomen circumference at pubic level, measured in centimeters., Circumference just below the buttocks in the sub-gluteal groove, measured in centimeters Right Thigh: Circumference just below the buttocks, measured in centimeters, Left Thigh: Circumference just below the buttocks, measured in centimeters, Right Arm: Circumference just below the armpit, measured in centimeters, Left Arm: Circumference just below the left armpit, measured in centimeters.

This information is collected in sheets 4, being these sheets powered by Google Sheets, MySQL tables or any other software adequate for such end, and then stored in a database 9. An automated Risk-Group classification process 5 is then performed on the stored data. This process consists of applying the formula “Risk score = W₁ f₁ + W₂ f₂+ W₃f₃+ W₄f₄= 1- 2.5” to the information collected from the patient, where W₁ is the age of the patient, W₂ is the BMI of the patient, W₃ is the Caprini Score of the patient, W₄ is the number of cigarettes the patient smokes per week, f₁ is 100^{∧}(-3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more.

Subsequently, a process of Cleaning and Filtering 6 is performed and lastly, the information retrieved in the Deployment of Information step 7, in which the Physician 2 can access the processed data including the risk factor and all the main insights and statistics generated by the system. An algorithm of Patient data error detection is continuously performed throughout the process. Periodically, a Reassessment process 10 is also performed, whenever new patient information is available, there are data changes in patient data and/or in the risk factors’ incidence levels or known correlations.

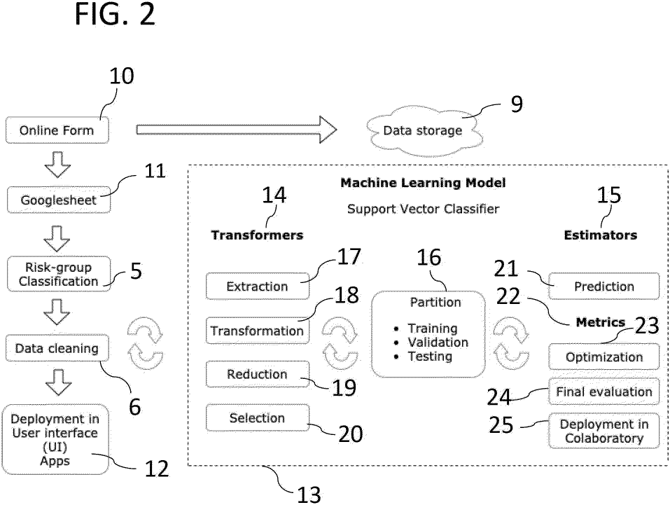

In FIG. 2, the Online Form 10 submits the collected data to a Google sheet 11 and stored in the data storage 9. The Risk Group classification 5 is performed based on this data which, after data cleaning 6, and deployed in a User Interface 12. A machine Learning model 13 is used for verifying the accuracy of the model and improving its predictive value based on the newly collected information. This model includes Transformers 14 and estimators 15. The transformers 14 include the extraction 17, Transformation 18, Reduction 19, and selection 20. A Partition 16 performs training, validation, and testing. The Estimators 15 include Prediction 21, the Metrics 22 used, optimization 23, final evaluation 24 and deployment in Collaboratory 25.

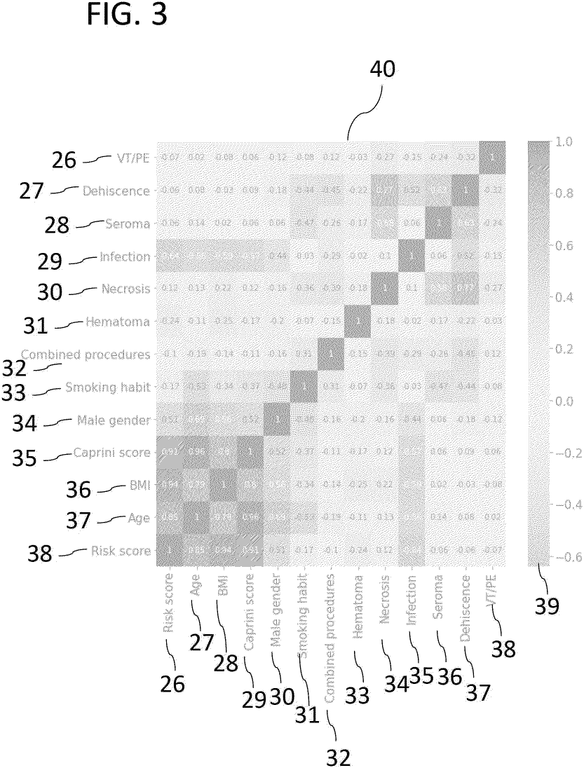

FIG. 3 shows the Patient’s collected data on a table having the different risk factors both as columns and rows to check their correlation. The numbers 40 in the interior of the table correspond to the correlation between the factors in the corresponding column and row. A color scale factor 39 is also shown, showing the strength of the correlations by color according to their relevance, for an easier visualization. The clinical data considered are: VT/PE 26, Dehiscence 27, Seroma 28, Infection 29, Necrosis 30, Hematoma 31, Combined Procedures 32, Smoking habit 33, male gender 34, Caprini Score 35, BMI 36, age 37 and Risk Score 38.

The formula “Risk score = W₁ f₁ + W₂ f₂+ W₃ f₃+ W₄ f₄ = 1- 2.5” , the variables selected being W₁ the age of the patient, W₂ the BMI of the patient, W₃ the Caprini Score of the patient, and W₄ the number of cigarettes the patient smokes per week, and the weighting factors being f₁ 100^{∧}(-3), f₂ 0.2 when W₂ is less than 25, 0.3 when W₂ is between 25 and 30, and 0.4 when W₂ is more than 30, f₃ 0.245, and f₄ 0.1 when W₄ is less than 7 and 0.3 when W₄ is 7 or more, have been developed as a conclusion of the studies performed with this method, for being highly relevant for risk prediction. However, variations in the variables used and their weighing factors, especially if obtained by the method hereby described, are encompassed within the spirit and scope of the present invention.

Finally, it should be noted that the above descriptions are based on data at the time of the invention, are dynamic, and are not intended to limit the invention. Especially the development of user interfaces, for example, applications and user interfaces that will change according to technological evolution, as well as medical recommendations that will change based on scientific evidence. Although the invention has been described in detail with reference to the above embodiments, it is still possible that the technical solutions described above will be modified, according to patient’s needs and preferences, and technological advances. For example, the use of the algorithm and system itself is not limited to plastic surgery and can be adapted to all surgical specialties. Any modification, replacement, improvement, etc. conducted within the spirit and principle of the invention will be included in the scope of protection of the invention.

1. A computing system for establishing a predictive score for the risk of complications after plastic surgery, said computing system comprising:
a. a patient interface;
b. a physician interface;
c. an online evaluation form accessible through said patient interface, said online evaluation form comprising a plurality of input-form-fields to enter information about a patient, said information comprising data needed to calculate a Caprini Score, cigarette consumption, BMI, and age of the patient;
d. a database in which said information is collected, stored, and from which said information can be retrieved;
e. an automated risk score calculation algorithm based on said information, using the following a risk score formula to calculate a Risk Score of the patient, said risk score formula being:
Risk Score = W1 f1 + W2 f2+ W3 f3+ W4 f4
Where: W1, W2, W3, and W4 are variables of the formula
f1, f2, f3, and f4 are factors of the formula
said variables and factors defined as:
W1 is "age of the patient", W2 is "BMI of the patient", W3 is "Caprini Score of the patient", W4 is "number of cigarettes the patient smokes per week", f1 is "100^(-3)", f2 is "0.2" when W2 is less than 25, "0.3" when W2 is between 25 and 30, and "0.4" when W2 is more than 30, f3 is "0.245", and f4 is "0.1" when W4 is less than 7 and "0.3" when W4 is 7 or more; and
f. a risk classification algorithm to assign a Risk Group to the patients based on said Risk Score, as “low risk” (when Risk Score is 1 to <1.2), “moderate risk” (when Risk Score is ≥1.2 to <1.4), and “high risk” (when Risk Score is ≥1.4) of complications;
wherein said computing system is embodied in a computer having a processor, a memory, a screen, and internet access.

2. The computing system for establishing a predictive score for the risk of complications after plastic surgery of claim 1, further comprising a deployment of information process to display said risk score and said risk group through said Physician’s interface.

3. The computing system for establishing a predictive score for the risk of complications after plastic surgery of claim 1, further comprising a system for fine-tuning said variables and said factors, by collecting clinical data, performing statistical analysis of data, using Pearson’s correlation coefficient test and p-value to find risk factors with statistically significant correlation with complications, performing a Pearson correlation coefficient test and a p-value between the risk score and the risk factors to determine which are independent predictors, establishing a predictive scoring system based on a multifactorial correlation analysis, as a Coefficient of multiple correlation, and validating said automated risk score calculation algorithm by machine learning using a vector classification system (VCS).

4. A method for establishing a scoring system and classification into risk groups for predicting the risk of complications after plastic surgeries comprising the steps of:
(1) Collecting information from a patient, said information comprising data needed to calculate a Caprini Score of the patient as well as cigarette consumption, BMI, and age of said patient, through an online evaluation form;
(2) Storing the information from step 1 on a database;
(3) Calculating the Caprini Score of the Patient;
(4) Calculating a Risk Score of the patient by applying a risk score formula, said risk score formula being “Risk score = W₁f₁ + W₂f₂+ W₃f₃+ W₄f₄= 1- 2.5” to the information collected from the patient in step 1, where W₁ is “age of the patient”, W₂ is “BMI of the patient”, W₃ is “Caprini Score of the patient”, W₄ is “number of cigarettes the patient smokes per week”, f₁ is 100^(-3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more;
(5) Classifying Patients in Risk Groups where three different risk groups are defined, said risk groups being: low risk when the Risk score is (1 to <1.2), moderate risk when the Risk score is (≥1.2 to <1.4), and high risk when the Risk score is (≥1.4); and
(6) displaying the Risk Score calculated on step 4 and the risk group calculated on step 5, together with the information collected on step 1, through a Physician’s interface in a computer or electronic device comprising a processor and a memory and connected to the Internet.

5. The method for establishing a scoring system and classification into risk groups for predicting the risk of complications after plastic surgeries of claim 4 further comprising the step of:
(7) calculating a risk-adjusted price (RAP) to measure a surgery price after taking into account said risk score, by defining a non-adjusted surgery price and then applying said risk score as a multiplier of this non-adjusted surgery price to obtain said RAP.
